(19) United States
(12) Patent Application Publication
(10) Pub. No.: US 2023/0054326 A1
(43) Pub. Date: Feb. 23, 2023

(54) PROBIOTIC COMPOSITION FOR TREATING RAPID EYE MOVEMENT SLEEP BEHAVIOR DISORDER, FORMULATION AND USE

(71) Applicant: BEIJING FRIENDSHIP HOSPITAL AFFILIATED TO CAPITAL MEDICAL UNIVERSITY, Beijing (CN)

(72) Inventors: Houzhen TUO, Beijing (CN); Yitong DU, Beijing (CN); Xiaojiao XU, Beijing (CN); Yue LI, Beijing (CN); Mingkai ZHANG, Beijing (CN); Ying CUI, Beijing (CN); Yun XUE, Beijing (CN); Dan GAO, Beijing (CN); Ting GAO, Beijing (CN); Zhi SHENG, Beijing (CN)

(73) Assignee: BEIJING FRIENDSHIP HOSPITAL AFFILIATED TO CAPITAL MEDICAL UNIVERSITY, Beijing (CN)

(21) Appl. No.: 17/938,671

(22) Filed: Oct. 6, 2022

Related U.S. Application Data
(63) Continuation-in-part of application No. PCT/CN2021/086312, filed on Apr. 10, 2021.

(30) Foreign Application Priority Data
Apr. 10, 2020 (CN) 202010281549.8

(51) Int. Cl. A61K 35/741; A61P 25/14

(52) U.S. Cl. CPC A61K 35/741; A61P 25/14

(57) ABSTRACT

A probiotic composition for treating rapid eye movement sleep behavior disorder, is prepared from Bacillus licheniformis, Bifidobacterium longum, Lactobacillus acidophilus, Enterococcus faecalis, and pharmaceutically acceptable adjuvants. The probiotic composition can improve the symptoms of rapid eye movement sleep behavior disorder of a patient, improve RBD symptoms of idiopathic RBD patients and PD patients, improve the motor symptoms and reduce the daily levodopa equivalent dose in PD patients.

### BACKGROUND

### Technical Field

The present disclosure relates to a probiotic composition, specifically to a probiotic composition for treating rapid eye movement sleep behavior disorder and a formulation obtained from the composition.

### Related Art

Rapid eye movement sleep behavior disorder (RBD for short) refers to a sleep disorder that occurs when a patient enters a period of rapid eye movement sleep, and during which the shouting, talking, or physical activity that accompanies dreams occurs since the muscles are not fully relaxed. The most common behaviors are waving arms and striking, kicking legs, talking, shouting, and getting up in bed, as well as grinding teeth, laughing, singing, talking on the phone, and even falling out of bed. These patients usually need to be treated with drugs. The most effective therapeutic drugs currently available include clonazepam, melatonin, and other medications. However, clonazepam has significant side effects and may also cause cognitive disorder with long-term application. Patients with idiopathic RBD (iRBD) are at high risk of developing neurodegenerative diseases such as Parkinson's disease, dementia with Lewy body, and multiple system atrophy after several years. 44 patients were followed up by foreign scholars with 45% being developed neurodegenerative disease after 5 years, and 82% being converted to neurodegenerative disease by 12 years, mostly to Parkinson's disease and dementia. Even those patients without a diagnosis of neurodegenerative disease showed abnormalities in biomarkers suggestive of Parkinson's disease or dementia risk.

Other disorders that present with symptoms of rapid eye movement sleep behavior disorder include Parkinson's Disease (PD for short). PD is a disease characterized by motor bradykinesia, static tremor, and rigidity, as well as postural instability. The main pathological features are the loss of dopaminergic neurons in the midbrain substantia nigra and the formation of Lewy bodies (mainly misfolded alpha-synuclein) in the residual neuronal cytoplasm. In addition to the above motor symptoms, the clinical manifestations include many non-motor symptoms such as constipation, rapid eye movement sleep behavior disorder (RBD for short), hyposmia, cognitive disorder, depression, etc. RBD is a common non-motor symptom of PD and occurs in more than half of patients with Parkinson's disease. It is characterized by the disappearance of skeletal muscle retardation and dream enactment behavior during rapid eye movement sleep. Dreams are often unpleasant, with contents such as being attacked, chased, and threatened, and behavioral disorders mainly manifests as shouting, murmuring, rapid and brief physical movements, punching and kicking, falling out of bed, etc.

In addition, the main manifestations of Parkinson's disease are movement disorders dominated by bradykinesia, static tremor, and rigidity, and bradykinesia is the core symptom. Patients often present with slow movements, shuffling gait, panicked gait, frozen gait, postural balance disorders, falls, etc. The current dopaminergic drugs for Parkinson's disease mainly address the motor symptoms of patients. However, after applying for a long period, the efficacy of the drugs decreases, and most patients will experience end-of-dose phenomenon, symptom fluctuation, dyskinesia, on-off phenomenon, etc. When the drugs take effect, the patients are more flexible in movement than before (on period), but they will have too much movement, which is manifested by non-stop twisting of trunk and limbs (dyskinesia). When the drugs are metabolized out of the body and thus have decreased efficacy, the patients have increased bradykinesia, even cannot move (off period), and they will need to take the drugs again and wait for the drugs to take effect before they can move again. A day in the life of a patient in the middle or late stages is a cycle of non-stop on-off periods, which is unbearably miserable. Moreover, some patients also experience aggravation of RBD and even hallucinations, and sleep disorders further lead to aggravation of clinical symptoms of PD, causing a vicious circle and greatly increasing the caregiving pressure on family members.

PD is a chronic progressive disease, and the current therapeutic drugs for PD motor symptoms mainly include the following classes: drugs that mainly improve the level of dopaminergic transmitters in the synaptic gap of neurons, including levodopa formulations, dopamine receptor agonists, monoamine oxidase inhibitors, COMT inhibitors, etc.; and drugs that modulate the action of other transmitters such as acetylcholine or NMDA, including benzhexol hydrochloride, amantadine, etc. These drugs can only control motor symptoms and cannot slow down the progression of the disease. Moreover, with the progression of the disease and long-term drug treatment, the dosage and types of the drugs for the patients with Parkinson's disease will gradually increase, but the efficacy will decline, and there will be motor complications such as “motor fluctuations”, “dyskinesia”, and “on-off phenomenon”. In addition, drugs used for treating PD can effectively improve the motor symptoms of Parkinson's disease, but cannot improve the symptoms of RBD simultaneously, and some drugs even aggravate the frequency of attack of RBD. Therefore, multiple drugs are needed to treat motor symptoms and RBD simultaneously. Currently, national guidelines recommend clonazepam for RBD treatment, which may lead to cognitive decline in patients after long-term use. Moreover, it is prone to develop dependence and drug resistance, and has greater side effects.

### SUMMARY

The primary technical problem to be solved by the present disclosure is to provide a probiotic composition for treating rapid eye movement sleep behavior disorder, which has been confirmed by clinical trials to have good therapeutic efficacy in the patients with idiopathic RBD and good stability and persistence of efficacy. The therapeutic effect on idiopathic RBD may delay the transformation of prodromal phase to clinical phase of Parkinson's disease. The probiotic composition also has good therapeutic effect on RBD symptoms in the patients with Parkinson's disease, and the motor symptoms are improved simutanously, which show that the probiotic composition may play a key protective role in the treatment of the patients with Parkinson's disease and has tendency reducing the daily levodopa equivalent dose for each in the patients.

Another technical problem to be solved by the present disclosure is to propose a probiotic formulation obtained by using the above probiotic composition.

A further technical problem to be solved by the present disclosure is to propose a new use of the above probiotic composition and formulation.

In order to achieve the above purpose, the present disclosure uses the following technical solutions:

A probiotic composition for treating rapid eye movement sleep behavior disorder, is prepared from Bacillus licheniformis, Bifidobacterium longum, Lactobacillus acidophilus, and Enterococcus faecalis.

The adjuvants used for preparing the probiotic composition in different product forms, with no effect on the efficacy of the probiotic itself, are also included. The adjuvants include, but are not limited to, prebiotics, dietary fiber, sugar additives, lipid additives, taste modifiers, etc. The adjuvants may also include, but are not limited to, solvents, emulsifiers, disintegrants, solubilizers, antioxidants, pH modifiers, osmolarity modifiers, bacteriostatic agents, diluents, wetting agents, binders, film-forming agents, etc.

Preferably, the viable count of Bacillus licheniformis in the probiotic composition is not less than 10.0×10⁸ CFU/g; the viable count of Bifidobacterium longum in the probiotic composition is not less than 4.0×10⁸ CFU/g; the viable count of Lactobacillus acidophilus in the probiotic composition is not less than 1.0×10⁸ CFU/g; and the viable count of Enterococcus faecalis in the probiotic composition is not less than 0.5×10⁸ CFU/g.

The above preferred range combined with the results of clinical trials can also be extended to the following range. The inventors conducted clinical trials using the probiotic composition within the following range and confirmed that the following range can also achieve the technical effect described in the present disclosure: the viable count of Bacillus licheniformis in the probiotic composition is not less than 1×10⁷ CFU/g; the viable count of Bifidobacterium longum in the probiotic composition is not less than 1×10⁷ CFU/g; the viable count of Lactobacillus acidophilus in the probiotic composition is not less than 1×10⁷ CFU/g; and the viable count of Enterococcus faecalis in the probiotic composition is not less than 1×10⁷ CFU/g.

The above viable count is the amount in the dried bacterial powder composition.

The viable count of the probiotic composition according to the present disclosure can be controlled within the above range, but is not limited to the above range in the actual manufacturing and application process. The viable count in the probiotic composition only needs to meet the national standards of the industry, and can achieve good colonization in the user's intestinal tract, and each strain can have a good synergistic effect with each other.

According to the above probiotic composition for treating rapid eye movement sleep behavior disorder, preferably, the probiotic composition is obtained by preparing the above composition as a lyophilized powder and then mixing.

Use of the above probiotic composition in the preparation of probiotic formulations, drugs, nutraceutical supplements and/or food products for the treatment of rapid eye movement sleep behavior disorder is included. The rapid eye movement sleep behavior disorder includes, but is not limited to, idiopathic rapid eye movement sleep behavior disorder and/or rapid eye movement sleep behavior disorder in Parkinson's disease.

A probiotic formulation for treating rapid eye movement sleep behavior disorder, is prepared from the above probiotic composition and adjuvants. The adjuvants are auxiliary ingredients required to prepare the probiotic composition into different probiotic dosage forms, and the adjuvants include, but are not limited to solvents, emulsifiers, disintegrants, solubilizers, antioxidants, pH modifiers, osmolality modifiers, diluents, wetting agents, binders, film-forming agents, etc. The adjuvants also include, but are not limited to, prebiotics, dietary fiber, sugar additives, lipid additives, taste modifiers, etc.

A method for preparing a probiotic formulation for treating rapid eye movement sleep behavior disorder includes the following steps:

(1) preparing each bacterial powder: preparing dried bacterial powder of the above Bacillus licheniformis, Bifidobacterium longum, Lactobacillus acidophilus, and Enterococcus faecalis, respectively; and

(2) mixing each dried bacterial powder obtained in step (1) with the adjuvants acceptable in the art to prepare a final dosage form.

The adjuvants described in the above step (2) can be selected according to the prepared dosage form without specific limitations, such as starch, dextrin, lactose, microcrystalline cellulose or inorganic salts, etc. for the solid formulations; starch slurry, mucilage, etc. for the wetting agents and binders; dry starch, sodium carboxymethyl starch, effervescent disintegrant, etc. for the disintegrants; and enteric coating, etc. for the film coating.

Use of the probiotic formulation prepared by the above method in the preparation of drugs, nutraceutical supplements and/or food products for the treatment of rapid eye movement sleep behavior disorder is included. The rapid eye movement sleep behavior disorder includes, but is not limited to, rapid eye movement sleep behavior disorder in Parkinson's disease and idiopathic rapid eye movement sleep behavior disorder.

A food composition or supplement for treating rapid eye movement sleep behavior disorder is included.

The probiotic composition is prepared from Bacillus licheniformis, Bifidobacterium longum, Lactobacillus acidophilus, Enterococcus faecalis, and pharmaceutically acceptable adjuvants, wherein

the viable count of Bacillus licheniformis in the probiotic composition is not less than 1×10⁷ CFU/g;

the viable count of Bifidobacterium longum in the probiotic composition is not less than 1×10⁷ CFU/g;

the viable count of Lactobacillus acidophilus in the probiotic composition is not less than 1×10⁷ CFU/g; and

the viable count of Enterococcus faecalis in the probiotic composition is not less than 1×10⁷ CFU/g.

Use of the above food composition or supplement in the preparation of nutraceutical supplements and/or food products for treating rapid eye movement sleep behavior disorder in Parkinson's disease is included.

The types of the above food products include, but are not limited to cookies, dairy products, meal replacements, meat products, sauces, ice cream, fermented products, fruit juices, rice wine, confectionery, canned food, pickled products, condiments, soy products, chocolate, fillings, tea products, puffed food, etc. The food product may also be a food additive.

The types of the above nutraceutical supplements include, but are not limited to medicinal wine, capsules, tablets, electuary, tea products, oral liquid, granules, fermented products, honey cream, distillate medicinal water, powder, meal replacements, etc. The nutraceutical may also include a nutraceutical additive.

The Bacillus licheniformis, Bifidobacterium longum, Lactobacillus acidophilus, Enterococcus faecalis used in the present disclosure are strains well known to those skilled in the art. The lyophilized powder of the strains according to the present disclosure is commercially available, which only needs to meet the requirements for the live bacteria according to the present disclosure, and can be compounded, mixed and dispensed in accordance with methods well known in the art. The probiotic composition can also be combined with acceptable adjuvants to prepare different products such as powders, granules, tablets, capsules, based on well-known methods for preparing drugs, nutraceuticals and foods. The live probiotic bacteria used in the present disclosure can also be obtained by the culture method well known in the art. The method for preparing probiotic and the compounding method are not limited to what is disclosed in the present invention, and any conventional and well-known method can be used in the present disclosure.

The above culture methods include, but are not limited to liquid fermentation method, solid fermentation method, and semi-solid fermentation method which are well known.

Types of the above liquid fermentation method include, but are not limited to, batch fermentation, continuous fermentation, and fed-batch fermentation. A medium used in the liquid fermentation method includes, but is not limited to LB medium, AAM-AS liquid medium, MRS medium, PTYG medium, PY medium, YPD medium, BSM medium; and the improved, modified, and optimized types thereof.

The types of the above solid fermentation method include, but are not limited to natural enrichment solid-state fermentation, intensified mixed microbial solid-state fermentation, limited mixed microbial solid-state fermentation, and single bacteria solid-state pure-fermentation. The medium used in the solid fermentation method includes, but is not limited to LB solid medium, YEB solid medium, Sabouraud's agar medium, R2A agar medium, YPD solid medium, BSM solid medium; and the improved, modified, and optimized types thereof.

Types of the above semi-solid fermentation method include, but are not limited to batch fermentation. The medium used in the semi-solid fermentation method includes, but is not limited to LB medium, YEB medium, Sabouraud's agar medium, R2A agar medium, YPD medium; and the improved, modified, and optimized types thereof.

The use method recommended by the present disclosure for patients with rapid eye movement sleep behavior disorder is as follows: the probiotic capsule according to the present disclosure is 250 mg per capsule, where the viable count in the capsules shall meet the requirements according to the present disclosure, the weight of the probiotic lyophilized powder contained in the capsule is related to the preparation method of the dry powder, and a slight difference is feasible.

The use method recommended by the present disclosure for patients with Parkinson's disease is as follows: oral administration, two capsules each time, and three times a day. It can also be increased or decreased according to the patient's condition.

Advantages and beneficial effects according to the present disclosure are as follows:

The probiotic composition according to the present disclosure and the probiotic formulation components prepared from the composition act synergistically, and have been confirmed in clinical trials to be effective in treating and improving RBD symptoms in patients, with good persistence and stability for the treatment effect of RBD symptoms. The present disclosure also has a therapeutic effect on the RBD symptoms of PD patients, and can effectively improve the motor symptoms of PD patients, solving the problem that the related art drugs cannot improve the treatment of motor symptoms and non-motor symptoms in PD patients at the same time. The probiotic formulation according to the present disclosure has no side effects, does not produce drug dependence and drug resistance, and can effectively reduce the dosage of levodopa in patients with PD, avoiding patients to develop resistance to levodopa prematurely, which has a better protective effect on patients. The present disclosure also has a significant improvement effect on the RBD-HK score of idiopathic rapid eye movement sleep behavior disorder (iRBD), and combined with the research results in PD patients, shows that the probiotic composition according to the present disclosure can significantly improve both RBD in Parkinson's disease and idiopathic RBD, suggesting that the probiotic composition provided by the present disclosure can be applied to the prodromal phase in Parkinson's disease and is expected to delay the transformation of prodromal phase to clinical phase of Parkinson's disease.

### BRIEF DESCRIPTION OF THE DRAWINGS

FIG. 1 is graph showing the changes of RBD-HK scores in three groups of RBD+PD patients.

FIG. 2 is graph showing the changes of UPDRS-III scores from baseline in three groups of RBD+PD patients after 12 and 24 weeks of treatment.

FIG. 3 is graph showing the comparison of the improvement rate of motor symptoms in three groups of RBD+PD patients after 12 weeks of treatment.

FIG. 4 is graph showing the comparison of the improvement rate of motor symptoms in three groups of RBD+PD patients after 24 weeks of treatment.

FIG. 5 is graph showing the variation trend of levodopa equivalent dose in three groups of RBD+PD patients.

FIG. 6 is graph showing the comparison of RBD-HK scores before and after iRBD treatment in efficacy trial 2 of the present disclosure.

FIG. 7 is graph showing the comparison of RBD-HK scores before and after iRBD intervention in two groups in the efficacy trial 3 of the present disclosure.

### DETAILED DESCRIPTION

The inventors have confirmed through clinical trials that the combination of Bacillus licheniformis, Bifidobacterium longum, Lactobacillus acidophilus, and Enterococcus faecalis has a better efficacy on the RBD symptoms in patients with idiopathic RBD and patients with Parkinson's disease, with better persistence and stability of efficacy, and has a protective effect on the disease progression in the patients with Parkinson's disease. The present disclosure has no specific requirements on the viable count of the above four strains, and only needs to meet the local production standards of the probiotic products and can be colonized in the intestinal tract of patients. The viable count of each strain described in the specific embodiments of the present disclosure is a recommended and better solution.

The specific embodiments are as follows.

The lyophilized probiotic powder as raw materials used in the examples of the present disclosure are commercially available.

In addition to being purchased commercially, the lyophilized probiotic powder can be prepared by the related art, and the specific preparation method can include the following steps:

(1) The seed strain in the seed medium is inoculated to obtain the inoculated product.

(2) The inoculated product in step (1) is placed in a fermentation medium for fermentation, and the pH remains unchanged during the fermentation to obtain a fermentation product.

(3) The fermentation product obtained in step (2) and a protective agent are lyophilized to obtain a lyophilized bacterial powder.

The above strains are Bacillus licheniformis, Bifidobacterium longum, Lactobacillus acidophilus, or Enterococcus faecalis.

The above protective agent is a well-known ingredient in the art and can be selected from skimmed milk powder, allolactose, Vc-Na, starch, sodium glutamate, etc.

The above culture for seed strain is a conventional technique, which can be found in “Studies on Formation of Lactic Acid and Growth of Bacillus Licheniformis by Biotin Variation” (Chinese Journal of Biotechnology, January 1998), “Optimization of Bifidobacterium Longum Enrichment Medium” (Science and Technology of Food Industry, No. 04, 2004), “Optimization of Lactobacillus Acidophilus Enrichment Medium” (Science and Technology of Food Industry, No. 06, 2002), and “Study on Health Care Yogurt Starter for Lactobacillus Acidophilus and Streptococcus Faecalis” (Journal of Anhui Agricultural Technology Normal University, No. 01, 2000).

The above fermentation culture is a conventional technique, and reference can be made to the related art: “Optimization of Bacillus Licheniformis 10236 Fermentation Conditions” (Journal of Hebei Agricultural Science, No. 05, 2019), “Optimization of Liquid Fermentation Conditions and Determination of Growth Curve for Bifidobacterium Longum” (Preparation and Technology, Vol. 9, No. 31, November 2012”), “Development of Lyoprotectant for Lactobacillus Acidophilus and Derived Direct-Vat-Set Composite Culture” (Modern Food Science Technology, 2019, 35(09):248-257.), and “Optimization of Encapsulated Fermentation Medium for Enterococcus Faecalis by Response Surface Method” (Science and Technology of Food Industry, No. 21, 2019)

The method for preparing the lyophilized bacterial powder is a conventional technology, and reference may be made to well-known technologies such as CN110367543A, CN110720638A, and CN102356912B.

The probiotic composition, preparation method and colony standard of the present disclosure all conform to Chinese Pharmacopoeia, Edition 2010, Part III and Chinese Biological Products Regulation, Edition 2000.

The CFU/g in the present disclosure means the number of colonies contained in per g of the tested sample, and CFU is the colony forming unit. The CFU/g in the following examples generally refers to “equal to” or “not less than”. In actual use, in order to ensure the viable count within the effective range during the shelf life and the survival rate of strains in the intestinal tract of patients after administration, the viable count in the product is generally “not less than”.

Probiotic capsules are prepared in the following examples of the present disclosure, and the equipment as used includes a three-dimensional motion mixer (SBH series, Jiangsu Guibao Group Co., Ltd.) and a capsule filling machine (Beijing Guowei Hejiao Medical Equipment Co., Ltd.). The probiotic composition of the present disclosure can also be prepared as powders, granules or tablets by those skilled in the art, if needed. The adjuvants used in each dosage form can be determined and prepared according to common knowledge in the art. The dosage forms, adjuvants and preparation methods do not affect the final technical effect of the present disclosure, and the type of adjuvants, the amount of adjuvants, the proportional relationship between adjuvants and the preparation method are not intended to limit the protection scope of the present disclosure. Of course, according to the actual situation, it is possible to choose not to prepare by oneself. A triple viable formulation available in the art that meets the viable count described in the present disclosure, such as Bifidobacterium longum, Lactobacillus acidophilus and Enterococcus faecalis triple viable formulation, and a Bacillus licheniformis viable capsule that meets the viable count described in the present disclosure can be chosen, and the combination of the two can also achieve the technical effect of the present disclosure.

### EXAMPLE 1 PREPARATION OF THE PROBIOTIC FORMULATION IN THE PRESENT DISCLOSURE

(1) Dried and lyophilized bacterial powders of Bacillus licheniformis, Bifidobacterium longum, Lactobacillus acidophilus, and Enterococcus faecalis and adjuvants were mixed to obtain a homogeneous mixture; and the adjuvants were pre-gelatinized starch and lactose in this example.

(2) The mixture obtained in step (1) were dispensed into capsules to obtain capsule formulation, in which the capsules of the capsule formulation were enteric capsules and each capsule contained 250 mg of mixed bacterial powder.

Each capsule contained Bacillus licheniformis of not less than 10×10⁸ CFU/g, Bifidobacterium longum of not less than 4.0×10⁸ CFU/g, Lactobacillus acidophilus of not less than 1.0×10⁸ CFU/g, and Enterococcus faecalis of not less than 0.5×10⁸ CFU/g.

After satisfying the above viable counts, the remainder was the adjuvant and the mass ratio of pre-gelatinized starch to lactose was 1:1.

### EXAMPLE 2 PREPARATION OF THE PROBIOTIC FORMULATION IN THE PRESENT DISCLOSURE

(1) Dried and lyophilized bacterial powders of Bacillus licheniformis, Bifidobacterium longum, Lactobacillus acidophilus, and Enterococcus faecalis and adjuvants were mixed to obtain a homogeneous mixture; and the adjuvants were pre-gelatinized starch and lactose in this example.

(2) The mixture obtained in step (1) were dispensed into capsules to obtain capsule formulation, in which the capsules of the capsule formulation were enteric capsules and each capsule contained 250 mg of mixed bacterial powder.

Each capsule contained Bacillus licheniformis of not less than 10×10⁹ CFU/g, Bifidobacterium longum of not less than 4.0×10⁹ CFU/g, Lactobacillus acidophilus of not less than 1.0×10⁹ CFU/g, and Enterococcus faecalis of not less than 0.5×10⁹ CFU/g.

After satisfying the above viable counts, the remainder was the adjuvant.

### EXAMPLE 3 PREPARATION OF THE PROBIOTIC FORMULATION IN THE PRESENT DISCLOSURE

(1) Dried and lyophilized bacterial powders of Bacillus licheniformis, Bifidobacterium longum, Lactobacillus acidophilus, and Enterococcus faecalis and adjuvants were mixed to obtain a homogeneous mixture; and the adjuvants were pre-gelatinized starch and lactose in this example.

(2) The mixture obtained in step (1) were dispensed into capsules to obtain capsule formulation, in which the capsules of the capsule formulation were enteric capsules and each capsule contained 250 mg of mixed bacterial powder.

Each capsule contained Bacillus licheniformis of not less than 10×10¹⁰ CFU/g, Bifidobacterium longum of not less than 4.0×10¹⁰ CFU/g, Lactobacillus acidophilus of not less than 1.0×10¹⁰ CFU/g, and Enterococcus faecalis of not less than 0.5×10¹⁰ CFU/g.

After satisfying the above viable counts, the remainder was the adjuvant.

### EXAMPLE 4 PREPARATION OF THE PROBIOTIC FORMULATION IN THE PRESENT DISCLOSURE

(1) Dried and lyophilized bacterial powders of Bacillus licheniformis, Bifidobacterium longum, Lactobacillus acidophilus, and Enterococcus faecalis and adjuvants were mixed to obtain a homogeneous mixture; and the adjuvants were pre-gelatinized starch and lactose in this example.

(2) The mixture obtained in step (1) were dispensed into capsules to obtain capsule formulation, in which the capsules of the capsule formulation were enteric capsules and each capsule contained 250 mg of mixed bacterial powder.

Each capsule contained Bacillus licheniformis of not less than 1×10⁷ CFU/g, Bifidobacterium longum of not less than 1×10⁷ CFU/g, Lactobacillus acidophilus of not less than 1×10⁷ CFU/g and Enterococcus faecalis of not less than 1×10⁷ CFU/g.

After satisfying the above viable counts, the remainder was the adjuvant.

### Efficacy Trial 1 of the Present Disclosure—Parkinson's Disease Cohort Study

### I. Materials and Methods

a) Subjects

1. Inclusion criteria:

To be eligible for this study, subjects must meet all of the following inclusion criteria:

1) Those who were aged 40 to 80 years old, male or female;

2) Patients with primary Parkinson's disease, were in line with the MDS clinical diagnostic criteria for Parkinson's disease (2015); modified Hoehn and Yahr grading of Parkinson's disease ≤3; no dementia, Mini-mental State Examination (MMSE) score >24;

3) The motor symptoms and RBD symptoms of Parkinson's disease were stable for 1 month before enrollment, and all administration was not adjusted for 1 month before enrollment; no clonazepam or melatonin or other benzodiazepines were administrated for 1 month before enrollment;

4) No intestinal probiotics or prebiotics (including lactulose) or antibiotics were administrated for 2 months before enrollment; if administrated, a 2-month washout period should be performed; and 5) Those who understood and agreed to follow the research protocol, and agreed to enroll and sign the informed consent form.

2. Exclusion criteria:

Subjects who meet any of the following criteria are not eligible for this study:

1) Those who had Parkinsonism-plus syndrome and secondary Parkinson's syndrome, such as multiple system atrophy, progressive supranuclear palsy, corticobasal degeneration, dementia with Lewy body, vascular Parkinson's syndrome, postencephalitic Parkinsonism, or any other non-primary Parkinson's disease;

2) Any probiotics or prebiotics (including lactulose) or antibiotics were administrated within 2 months before enrollment;

3) Those who had the following diseases: such as Alzheimer's disease, malignant tumor, myelopathy, epilepsy, obstructive sleep apnea syndrome, autonomic nervous disease (urinary retention, urinary incontinence or orthostatic hypotension, blood pressure drop of more than 30/15 mmHg after standing for 5 minutes), etc.; new-onset cerebrovascular disease or severe sequelae of cerebrovascular disease affecting the assessors within 3 months;

4) Those who had history of gastrointestinal tumor, inflammatory bowel disease, and other acute and chronic inflammations of the gastrointestinal tract within 3 months (including acute exacerbation of cholecystitis);

5) Those who had severe cardiovascular disease (such as American Heart Association function class III-IV congestive heart failure, history of myocardial infarction within 6 months, etc.);

6) Those who had severe liver and kidney dysfunction, glutamic-pyruvic transaminase, glutamic oxalacetic transaminase, and total bilirubin of higher than 1.5 times the upper limit of normal; serum creatinine of 1.5 times higher than the upper limit of normal;

7) Those who were pregnant and lactating women or women of reproductive age between 40 and 60 years old with positive HCG;

8) Those who were known to be allergic to the test drug or related products;

9) Those who had a history of drug abuse or alcohol dependence;

10) Those who participated in other clinical trials within 3 months before enrollment; and

11) Those who refused to be enrolled and cannot cooperate with the researcher; and those who were judged by the researcher to be unsuitable for enrollment.

3. Randomization scheme: this study was a prospective open cohort study. According to whether the patients had RBD, the patients were further stratified into RBD+PD patients and RBD−PD patients, and the patients in each stratum were randomly divided into test group 1, test group 2 and control group.

RBD+ criteria (Stiasny-Kolster K, Mayer G, Schafer S, et al. The REM sleep behavior disorder screening questionnaire—a new diagnostic instrument [J]. Mov Disord, 2007, 22(16): 2386-2393. Shen S S, Shen Y, Xiong K P, Chen J, Mao C J, Huang J Y, Li J, Han F, Liu C F, Validation study of REM sleep behavior disorder questionnaire-Hong Kong (RBDQ-HK) in east China. Sleep Med. 2014 Aug; 15(8):952-8. doi: 10.1016/j.sleep.2014.03.020.): For Rapid eye movement sleep behavior disorder screening questionnaire (RBDSQ), RBDSQ>5, for Rapid eye movement sleep behavior disorder questionnaire Hong Kong (RBD-HK for short), RBD-HK score ≥17, and RBD is diagnosed by polysomnography; and those who did not meet the above criteria were assigned as RBD−.

4. Drop out, discontinuation and withdrawal scheme: patients who cannot continue to participate in this study, or patients who cannot be contacted during multiple follow-up are considered as drop out; those who developed any inflammation required to be treated with antibiotics during the observation period will have unaccepted scale scores during the use period and are considered as discontinuation; and those who developed acute cardiovascular and cerebrovascular accidents or other serious adverse events are considered as withdrawal.

### II. Test Method

a) Intervention samples: Test group 1 (intervention group): the probiotic formulation was obtained in Example 1 of the present disclosure. Test group 2 (intervention group): each capsule contained a probiotic capsule containing 1.0×10⁷ CFU of Bifidobacterium longum, 1.0×10⁷ CFU of Lactobacillus acidophilus, and 1.0×10⁷ CFU of Enterococcus faecalis (purchased from Shanghai Sine Pharmaceutical Laboratories Co., Ltd., National Medicine Permission Number S10950032). Control group: only pre-enrollment therapy thereof was maintained, and no probiotics or prebiotics were added.

b) Test method:

On the basis of conventional treatment of PD, test group 1 was given the formulation of Example 1, 2 capsules each time, three times a day. On the basis of conventional treatment of PD, test group 2 was given the formulation of test group 2 according to the instruction, 4 capsules each time, twice a day. The blank control group was only given conventional PD drugs.

The daily dose of all anti-PD drugs was converted into levodopa equivalent dose according to the following conversion formula: 100 mg levodopa=100 mg piribedil=1 mg pramipexole=5 mg ropinirole=10 mg selegiline=100 mg amantadine=133 mg carbidopa=1 mg rasagiline. The dose of entacapone was calculated as levodopa equivalent dose with a 33% potentiation.

After enrollment, RBD+PD patients were not given clonazepam or other benzodiazepines or melatonin or the like for sleep intervention until the end of the observation period.

c) Evaluation: All patients were collected for electrocardiogram, blood routine, liver and kidney function, and electrolyte testing at the time of enrollment and week 12. Rapid eye movement sleep behavior disorder screening questionnaire (RBDSQ) was used for screening, and rapid eye movement sleep behavior disorder questionnaire Hong Kong (RBD-HK) was used to assess RBD and the severity thereof; and Unified Parkinson's Disease Rating Scale (UPDRS for short) Part III UPDRS-III was used as an indicator for evaluating the severity of motor symptoms. The difference of the score at week 12 or week 24 minus the score at week 0 was used for statistics. The difference >3 was regarded as aggravation, the difference <−3 was regarded as improvement, and the difference between −3 and 3 was regarded as stability. The UPDRS-III score and RBD-HK score were completed at week 0, week 12, and week 24 after enrollment, respectively, and the names and doses of patients' conventional anti-PD drugs were recorded.

d) Data statistics: SPSS26.0 was used for statistical analysis of the data. Measurement data were expressed as mean±standard deviation (x±s), and enumeration data were expressed as percentage. The data at week 12 was used as the main statistical indicator. Paired t-test was used for intra-group comparison of measurement data; analysis of variance was used for comparison among three groups; analysis of variance was used for comparison among three groups at different time points; and chi-square test or Fisher's exact test was used for enumeration data. P<0.05 meant that the difference was statistically significant.

### III. Test Results and Analysis

### General Patient Information

This study was approved by Ethics Committee of Beijing Friendship Hospital, Capital Medical University.

Patients attending the neurology outpatient clinic and ward at Beijing Friendship Hospital, Capital Medical University from May 2018 to December 2019 were collected, all of whom signed an informed consent form.

A total of 62 patients with primary Parkinson's disease who met the inclusion criteria were collected, 20 in test group 1, 20 in test group 2, and 22 in control group with 2 drop-out cases. Finally, 60 patients were enrolled. 48 cases of RBD+ and 12 cases of RBD− were randomly assigned to three groups with 16 cases of RBD+PD patients and 4 cases of RBD−PD patients in each group. There were 35 males with a median age of 66 years, and there was no significant differences in age (P<0.05, analysis of variance) and gender (P<0.05, chi-square test) distribution between the three groups. There was no obvious abnormality in electrocardiogram, blood routine, liver and kidney function, electrolytes, etc. at the time of enrollment and week 12, and no patients withdrew from observation in advance due to the above reasons. No patients withdrew from observation due to serious adverse events. The H-Y grading was 2.10±0.68, 2.15±0.65, and 1.75±0.60 for the three groups of patients, respectively, with no statistically significant difference (P=0.110>0.05, analysis of variance).

### a) Comparison of RBD Effects

### 1. 12-Week Follow-Up Results

As shown in Table 1, at the time of enrollment, RBD-HK scores of 16 RBD+PD patients in all groups were 38.75±10.89 (test group 1), 33.19±12.50 (test group 2), and 30.56±12.79 (control group), respectively, with no statistically significant difference among three groups (P=0.178>0.05, analysis of variance). After 12 weeks of treatment, compared to week 0 in this group, the differences in RBD-HK scores were statistically significant in both test group 1 and test group 2 (p<0.001 (test group 1), p<0.05 (test group 2)), while there was no significant difference in the scores of the control group before and after enrollment (p>0.05). For the comparison of the changes in RBD-HK scores from baseline among three groups, the difference between the two groups was statistically significant (p<0.05); and the differences of changes in RBD-HK scores from baseline between test group 1 and control group (p<0.001), test group 2 and control group (p=0.025), and test group 2 and test group 1 (p=0.035) were statistically significant (p<0.05. analysis of variance).

TABLE 1
RBD-HK scores of patients with RBD +
PD in three groups at week 12

| | | | Change from |
|---|---|---|---|
| Group | Week 0 | Week 12 | baseline |
| Test group 1 | 38.75 ± 10.89 | 29.13 ± 12.46*** | −9.63 ± 6.1^{###} |
| (n = 16) | | | |
| Test group 2 | 33.19 ± 12.50 | 29 ± 12.90* | −4.19 ± 6.31^{#} |
| (n = 16) | | | |
| Control group | 30.56 ± 12.79 | 32.06 ± 11.97 | 1.5 ± 5.03 |
| (n = 16) | | | |

*P < 0.05,
**P < 0.01, and
***P < 0.001, compared to this group before treatment; and
^{#}P < 0.05,
## P < 0.01, and
^{###}P < 0.001, compared to the control group after treatment.

### 2. 24-Week Follow-Up RBD Results

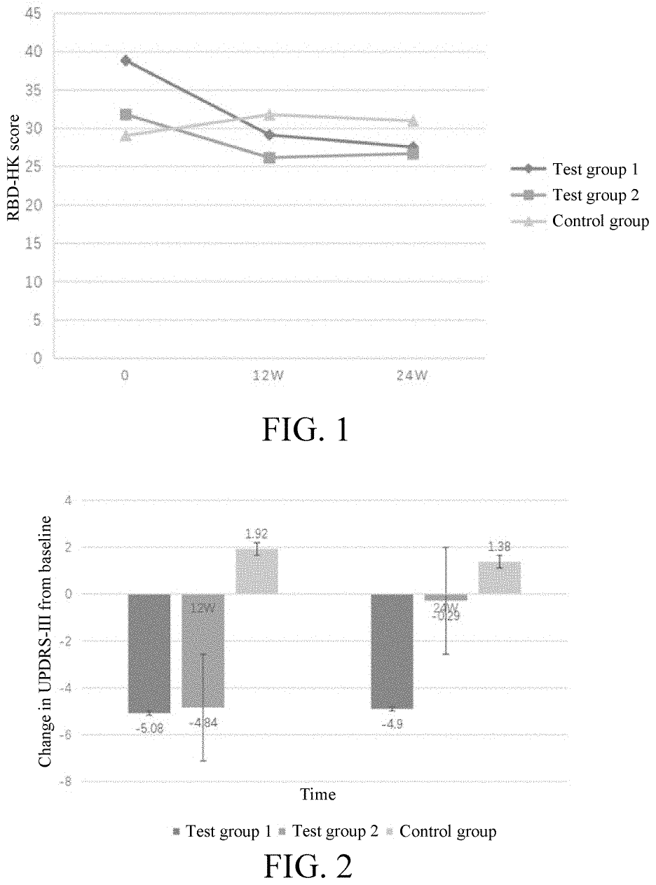

See Table 2 and FIG. 1 for details, 14, 11, and 14 cases in the three groups completed 24-week follow-up, respectively (reasons for drop out: some patients failed to meet the period for 24-week follow-up, and a small number of patients could not be followed up because of the current 2019-2020 COVID-19 pandemic). For the comparison of the changes in RBD-HK scores from baseline, there was a statistically significant difference among three groups (p<0.05); and the differences of changes in RBD-HK scores from baseline between test group 1 and control group (p<0.001), test group 2 and control group (p=0.021), and test group 1 and test group 2 (p=0.048) were statistically significant (p<0.05. analysis of variance).

TABLE 2
RBD-HK scores of patients with RBD + PD in three groups at week 24

| | | | Change from | | Change from |
|---|---|---|---|---|---|
| | | | baseline at | | baseline at |
| Group | Week 0 | Week 12 | week 12 | Week 24 | week 24 |
| Test group 1 | 38.86 ± 10.87 | 29.14 ± 12.23*** | −9.71 ± 6.20^{###} | 27.57 ± 11.29*** | −11.29 ± 7.44^{###} |
| (n = 14) | | | | | |
| Test group 2 | 31.82 ± 9.91 | 26.18 ± 10.46* | −5.64 ± 5.77 | 26.73 ± 10.20* | −5.09 ± 3.83 |
| (n = 11) | | | | | |
| Control group | 29.07 ± 11.71 | 31.79 ± 11.62* | 2.71 ± 3.90 | 31 ± 13.24* | 1.93 ± 5.99 |
| (n = 14) | | | | | |

*P < 0.05,
**P < 0.01,
***P < 0.001, compared to this group before treatment; and
# P < 0.05,
## P < 0.01, and
^{###}P < 0.001, compared to the control group at the same period.

In conclusion: the results suggest that there was a sustained reduction in RBD-HK score in test group 1 (the present disclosure group) over time, i.e., there was a sustained improvement in the patient's RBD condition; for test group 2 (tri-bacteria group), the patients' RBD condition improved at week 12 of treatment compared with that at the time of enrollment, but there was no further improvement after continued treatment; and the RBD-HK scores in the control group were not statistically different at weeks 0, 12, and 24, suggesting that anti-Parkinson's disease motor symptoms drugs had no significant effect on the patients' RBD condition. Further as shown in FIG. 1, the RBD-HK score of test group 1 continued to decrease with a significant and stable trend of decrease. Although there was a decrease in test group 2, there was an obvious upward trend in the later period. Although both groups had certain efficacy, the persistence and stability of the efficacy of test group 1 was more excellent than that of test group 2. To sum up, it is suggested that for the RBD symptoms of PD patients, the therapeutic effect of test group 2 was better than that of the control group, and the therapeutic effect of test group 1 of the present disclosure was better than that of test group 2 and much better than that of the control group; and there was a continuous improvement in the efficacy provided by the present disclosure with the extension of treatment time.

### b) Movement Symptom Data Statistics

1. Comparison of UPDRS-III scores:

Note: since this scale required patients to come to the hospital for a face-to-face assessment by physicians, some patients missed the assessment time point as they failed to arrive at the hospital within the specified time, and since some patients did not meet the period for the 24-week follow-up, the number of patients that were followed up was partially reduced.

Based on a paired t-test, 19, 16, and 18 patients in the three groups completed the 12-week follow-up of UPDRS scale, respectively, and there was no statistically significant difference among the baseline UPDRS III scores of the three groups (P=0.05, analysis of variance). For intra-group comparison of UPDRS-III scores at week 12 of treatment with week 0, there were statistically significant differences in both test group 1 (P=0.001) and test group 2 (P=0.029), while there was no statistically significant difference in the control group (P=0.111).

See Table 3 for comparison of the change in UPDRS III scores after 12 weeks of treatment and scores at week 0, the changes for the three groups were −5.08±5.82 (test group 1), −4.84±8.01 (test group 2), and 1.92±4.84 (control group), respectively, and the difference was statistically significant (p<0.001). The difference of change in UPDRS III scores between test group 1 and control group (p=0.004), and test group 2 and control group (p=0.009) was statistically significant (P<0.01); and there was no statistically significant difference in the change in UPDRS III score between test group 2 and test group 1 (p=1.000>0.05) (variance analysis). However, the change in scores of test group 1 was better improved than that of test group 2 after 12 weeks of treatment.

TABLE 3
UPDRS III score of PD patients in the
three groups at week 12 of treatment

| | | | Change from |
|---|---|---|---|
| Group | Week 0 | Week 12 | baseline |
| Test group 1 | 21.66 ± 8.70 | 16.58 ± 8.93** | −5.08 ± 5.82^{##} |
| (n = 19) | | | |
| Test group 2 | 21.78 ± 9.45 | 16.94 ± 7.48* | −4.84 ± 8.01^{##} |
| (n = 16) | | | |
| Control group | 15.08 ± 8.27 | 17 ± 8.70 | 1.92 ± 4.84 |
| (n = 18) | | | |

*P < 0.05,
**P < 0.01, and
***P < 0.001, compared to this group before treatment; and
# P < 0.05,
^{##}P < 0.01, and
### P < 0.001, compared to the control group after treatment.

As shown in Table 4 and FIG. 2, the number of patients who completed the 24-week follow-up of UPDRS score was 15, 7, and 8 in each group, respectively. Based on the statistics on these patients, it was found that for intra-group comparison of UPDRS-III scores at week 24 of treatment with week 0, there was a statistically significant difference in test group 1 (P=0.020), and no statistically significant difference in both test group 2 (P=0.907) and control group (P=0.546). For the change in UPDRS-III scores at week 24 from baseline, there was no statistically significant difference between the three groups (P=0.09>0.05). For the comparison of the change in UPDRS-III scores, there was no statistically significant difference between test group 1 and the control group (p=0.126), test group 2 and the control group (p=1.000), and test group 1 and test group 2 (p=0.435) (p>0.05) (variance analysis). However, the trend of improvement was more pronounced in test group 1 (−4.9±7.20) than in test group 2 (−0.29±6.18) and control group (1.38±6.13) for the change in UPDRS-III scores at week 24 of treatment.

TABLE 4
UPDRS III scores of PD patients in the three groups at week 24

| | | | Change from |
|---|---|---|---|
| Group | Week 0 | Week 24 | baseline |
| Test group 1 | 19.73 ± 8.06 | 14.83 ± 9.60* | −4.9 ± 7.20 |
| (n = 15) | | | |
| Test group 2 | 18.36 ± 6.87 | 18.07 ± 7.76 | −0.29 ± 6.18 |
| (n = 7) | | | |
| Control group | 16.5 ± 9.50 | 17.88 ± 6.56 | 1.38 ± 6.13 |
| (n = 8) | | | |

*P < 0.05,
**P < 0.01, and
***P < 0.001, compared to this group before treatment; and
# P < 0.05,
## P < 0.01, and
### P < 0.001, compared to the control group after treatment.

2. Improvement rate statistics:

According to the literature, a difference between baseline UPDRS-III score after treatment and that at the time of enrollment of ≤|±3| was defined as stable disease, a negative score of >3 was defined as improvement, and a positive score of >3 was defined as aggravation. Then, after 12 weeks of treatment, there was a statistically significant difference in the improvement rate among the three groups (P=0.008<0.01). See Table 5 for comparison of the efficacy at week 12 of treatment, there was a statistically significant difference (p<0.05) in the improvement rate between test group 1 and control group (p=0.004) and test group 2 and control group (p=0.013), and there was no statistically significant difference (p>0.05, variance analysis) in the improvement rate between test group 1 and test group 2 (p=0.691).

TABLE 5
Comparison of the therapeutic effects in the three treatment regimens at week 12

| | Therapeutic Effects (UPDRS) | | | Improvement |
|---|---|---|---|---|
| Group | Improvement | Stable disease | Aggravation | rate |
| Test group 1 | 10 | 8 | 1 | 52.6%^{##} |
| (n = 19) | | | | |
| Test group 2 | 9 | 5 | 2 | 56.3%^{#} |
| (n = 16) | | | | |
| Control group | 2 | 8 | 8 | 11.1% |
| (n = 18) | | | | |

*P < 0.05,
**P < 0.01, and
***P < 0.001, compared to this group before treatment; and
^{#}P < 0.05,
^{##}P < 0.01, and
### P < 0.001, compared to the control group in the same period after treatment.

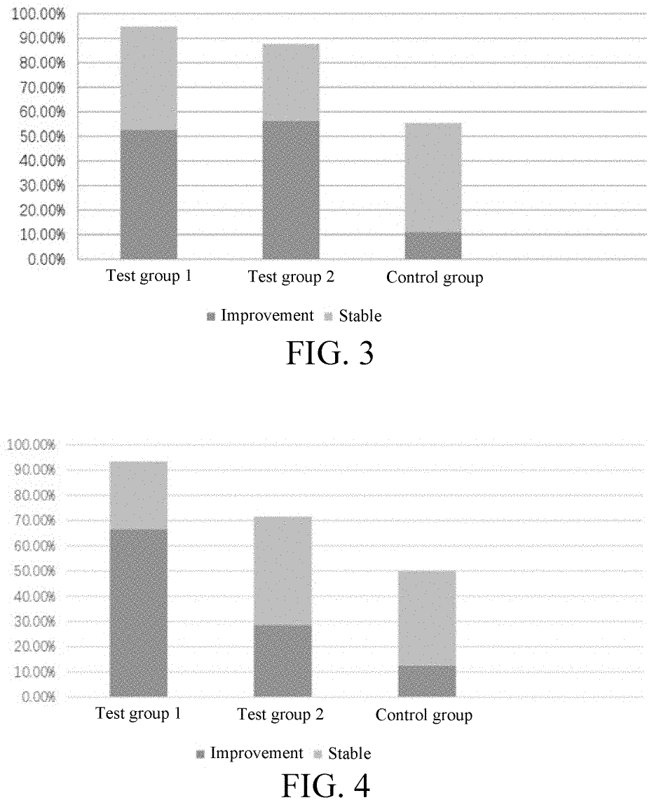

At week 24 of treatment, there was a statistically significant difference (p<0.05) in the improvement rate between test group 1 and control group (p=0.015), and there was no statistically significant difference (p>0.05, analysis of variance) in the improvement rate between test group 1 and test group 2 (p=0.198) and test group 2 and control group (p=0.814), and no statistically significant difference in the improvement rate among the three groups (p=0.062>0.05, analysis of variance). However, the improvement rate of 66.7% in test group 1 was significantly higher than that of 28.6% in test group 2. See Table 6 and FIG. 3.

TABLE 6
Comparison of the therapeutic effects in the three treatment regimens at week 24

| | Treatment Effectiveness (UPDRS-III) | | | Improvement |
|---|---|---|---|---|
| Group | Improvement | Stable disease | Aggravation | rate |
| Test group 1 | 10 | 4 | 1 | 66.7%^{#} |
| (n = 15) | | | | |
| Test group 2 | 2 | 3 | 2 | 28.6% |
| (n = 7) | | | | |
| Control group | 1 | 3 | 4 | 12.5% |
| (n = 8) | | | | |

*P < 0.05,
**P < 0.01, and
***P < 0.001, compared to this group before treatment; and
^{#}P < 0.05,
## P < 0.01, and
### P < 0.001, compared to the control group in the same period after treatment.

Referring to Table 6 and FIG. 4, the above results suggested that: after administrating probiotic treatment to the patients, both test group 1 and test group 2 significantly improved patients' motor symptoms, and test group 1 had a stronger improvement effect; and with the extension of treatment time, the treatment of motor symptoms was consistently effective in test group 1, the proportion of patients with improved symptoms gradually increased from 52.6% at week 12 to 66.7% at week 24, while the improvement rate in test group 2 (tri-bacteria group) decreased from 56.3% at week 12 to 28.6% at week 24, and the improvement rate in the control group was basically stable at 11.1% to 12.5%. The improvement rate at week 24 was significantly higher in test group 1 than in control group and test group 2. It showed that test group 1 of the present disclosure has a stronger tendency to improve the therapeutic effect of motor symptoms than test group 2, and the persistence and stability of the improvement trend were better in test group 1 than that in test group 2.

In conclusion: after administrating probiotic treatment to the patients, both the test group 1 of the present disclosure and the test group 2 improved the motor symptoms of the patients; compared with test group 2, the present disclosure was better than test group 2 in terms of UPDRS-III score and overall improvement rate; and with the extension of treatment time, the effect on improving motor symptoms gradually increased, and the overall improvement rate gradually increased. At week 24, 66.7% of patients showed significant improvement in motor symptoms, which was much higher than in test group 2 and control group. More importantly, the present disclosure showed a stronger, and more consistent and stable trend of improvement in motor symptoms than test group 2.

### c) Comparison of Levodopa Equivalent Dose

The statistical sample of patients in this group was the same as the statistical sample of patients with motor symptoms.

There was no significant difference in levodopa equivalent dose statistics among the three groups at the time of enrollment (P=0.227). For the comparison of change in levodopa equivalent dose at week 12 of treatment from baseline, there was a statistically significant difference among the three groups (P=0.004, <0.05). Both test group 1 and test group 2 showed a reduction in levodopa equivalent dose/day at week 12 compared to that at the time of enrollment, with a reduction of −22.37±45.56 mg/d and −8.59±42.50 mg/d, respectively. The levodopa equivalent dose reduction was statistically significant in test group 1 compared to control group (p=0.005) (p<0.05), and there was no statistically significant difference (p>0.05) in the change of levodopa equivalent dose between test group 2 and control group (p=0.051), and test group 1 and test group 2 (p=1.000) (analysis of variance). See Table 7.

TABLE 7
Comparison of levodopa equivalent dose of
PD patients in three groups at week 12

| | Levodopa equivalent dose (mg/d) | | Change from |
|---|---|---|---|
| Group | Week 0 | Week 12 | baseline |
| Test group 1 | 329.61 ± 196.57 | 307.24 ± 182.87* | −22.37 ± 45.56^{##} |
| (n = 19) | | | |
| Test group 2 | 437.5 ± 283.39 | 428.91 ± 262.04 | −8.59 ± 42.50 |
| (n = 16) | | | |
| Control group | 311.11 ± 171.03 | 348.61 ± 192.84* | 37.5 ± 69.79 |
| (n = 18) | | | |

*P < 0.05,
**P < 0.01, and
***P < 0.001, compared to before treatment; and
# P < 0.05,
^{##}P < 0.01, and
### P < 0.001, compared to the control group after treatment.

There was no statistically significant difference in the change in levodopa equivalent dose at week 24 of treatment among the three groups (P=0.22>0.05). There was no statistically significant difference (p>0.05) in the change in levodopa equivalent dose between test group 1 and control group (p=0.331), test group 2 and control group (p=0.491), and test group 1 and test group 2 (p=1.000) (analysis of variance). See Table 8.

TABLE 8
Levodopa equivalent dose of PD patients in three groups at week 24

| | | | Change from | | Change from |
|---|---|---|---|---|---|
| | Levodopa equivalent dose (mg/d) | | baseline at | | baseline at |
| Group | Week 0 | Week 12 | week 12 | Week 24 | week 24 |
| Test group 1 | 313.33 ± 197.46 | 286.67 ± 176.98 | −26.67 ± 48.71 | 285 ± 165.53 | −28.33 ± 78.41 |
| (n = 15) | | | | | |
| Test group 2 | 442.86 ± 326.70 | 428.57 ± 310.37 | −14.29 ± 22.59 | 414.29 ± 320.40 | −28.57 ± 39.34 |
| (n = 7) | | | | | |
| Control | 359.38 ± 135.75 | 378.13 ± 161.75 | 18.75 ± 42.85 | 378.13 ± 161.75 | 18.75 ± 45.81 |
| group | | | | | |
| (n = 8) | | | | | |

*P < 0.05,
**P < 0.01, and
***P < 0.001, compared to before treatment; and
# P < 0.05,
## P < 0.01, and
### P < 0.001, compared to the control group after treatment.

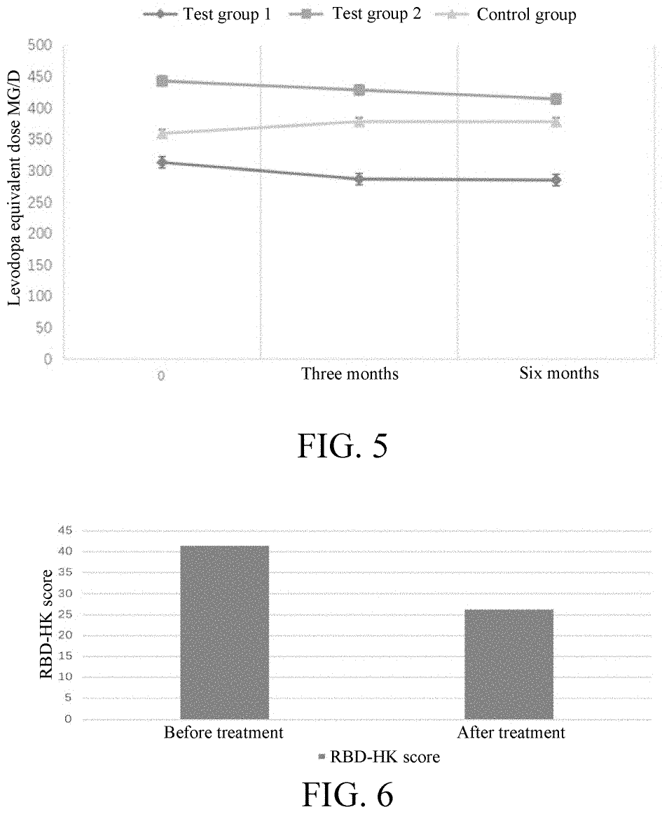

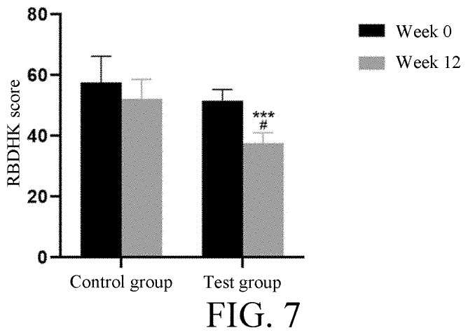

Patients who completed both the UPDRS III scores at weeks 12 and 24 and levodopa equivalent dose recordings were 15 in test group 1, 7 in test group 2, and 8 in control group, and the change in levodopa equivalent dose among the three groups was shown in FIG. 5.

In conclusion: after the treatment of test group 1 of the present disclosure, the patients' RBD severity was improved significantly, and the efficacy was significantly higher than that of test group 2; moreover, the improvement effect gradually increased over time; and along with the improvement of RBD, the patients' motor symptoms were significantly improved, and the improvement rate tended to increase significantly, while the daily levodopa equivalent dose was significantly reduced compared with that at the time of enrollment, suggesting that the improvement of motor symptoms resulted from probiotics rather than the increase of levodopa equivalent dose. There was a trend towards improved efficacy in test group 1 compared to test group 2.

The probiotic formulation prepared in Example 1 was used in the above clinical trial, and the same test conclusion can be achieved via testing the probiotic formulation prepared in Example 4 by the inventors. Theoretically, the technical effect of the present disclosure can be achieved by the probiotic having a viable count of not less than the CFU/g as described. Taking into account the loss rate of live bacteria during storage and transportation of probiotics, a slightly lower viable count than the limit of the present disclosure is also feasible.

### Efficacy Trial 2 of the Present Disclosure—Idiopathic RBD (iRBD) Cohort Study

### I. Materials and Methods

a) Subjects

1. Inclusion criteria:

To be eligible for this study, subjects must meet all of the following inclusion criteria:

1) Those who were aged 40 to 85 years old, male or female;

2) For Rapid eye movement sleep behavior disorder screening questionnaire (RBDSQ), RBDSQ>5, and for Rapid eye movement sleep behavior disorder questionnaire Hong Kong (RBD-HK for short), RBD-HK score ≥17; and

3) Diagnostic criteria for iRBD: Diagnosis was confirmed by a physician at Department of Neurology & Sleep Disorders Center based on polysomnography (PSG for short), where the patient had a history of dream enactment behavior, and video polysomnography showed increased electromyography activity or abnormal behavior during the rapid eye movement.

2. Exclusion criteria:

1) Those with a history of serious mental illness or drug abuse; a Hamilton Depression Scale score of ≥17, and/or an anxiety scale score of ≥14 or being treated with oral antidepressant or antipsychotics; being treated with oral clonazepam or other benzodiazepines and/or melatonin within 4 weeks; and 2) Those who had other central nervous system diseases, including cerebrovascular disease, brain tumors, inflammation, genetic metabolic diseases, traumatic brain injury, definite degenerative diseases of the central nervous system (e.g. Parkinson's disease, multiple system atrophy, dementia with Lewy body, etc.), and the like.

3. Drop out, discontinuation and withdrawal scheme: Patients who cannot continue to participate in this study, or patients who cannot be contacted during multiple follow-up are considered as drop out; those who developed any inflammation required to be treated with antibiotics during the observation period will have unaccepted scale scores during the use period and are considered as discontinuation; and those who developed acute cardiovascular and cerebrovascular accidents or other serious adverse events are considered as withdrawal.

b) Test method:

1. Intervention method: The probiotic formulation was obtained in Example 4 of the present disclosure, 2 capsules each time, three times a day, for 12 weeks of continuous intervention.

2. Evaluation: All patients were collected for electrocardiogram, blood routine, liver and kidney function, and electrolyte testing at the time of enrollment and week 12. Rapid eye movement sleep behavior disorder screening questionnaire (RBDSQ) was used for screening, and rapid eye movement sleep behavior disorder questionnaire Hong Kong (RBD-HK) was used to assess RBD and the severity thereof. 3. Data statistics: SPSS26.0 was used for statistical analysis of the data. Measurement data were expressed as mean±standard deviation (x±s), and the data at week 12 was used as the main statistical indicator. Paired t-test was used; and P<0.05 meant that the difference was statistically significant.

### II. Test Results and Analysis

This study was approved by Ethics Committee of Beijing Friendship Hospital, Capital Medical University.

Patients attending the neurology outpatient clinic and ward at Beijing Friendship Hospital, Capital Medical University from January 2019 to January 2021 were collected, all of whom signed an informed consent form.

A total of 6 iRBD patients, 5 males and 1 female, with a mean age of 64.7±2.7 years, who met the inclusion criteria and did not meet the exclusion criteria were collected. The RBDSQ scores of all patients were >5. The RBD-HK score before the intervention was 41.50±15.67. After 12 weeks of treatment, the RBD condition improved significantly in 5 patients and remained stable in 1 case, with a RBD-HK score of 26.17±9.99, which was statistically significantly different from that before the intervention (P=0.038, P<0.05), as shown in FIG. 6.

### III. In Conclusion

After the present disclosure was used for the treatment of idiopathic RBD (iRBD), most of the patients (5/6 cases) had a significantly improved RBD condition compared with that before treatment, and 1 case had a stable onset condition, suggesting that the present disclosure can significantly improve the symptoms of idiopathic RBD. Currently, no related reports have been seen. Idiopathic RBD is a prodromal manifestation of neurodegenerative diseases especially Parkinson's disease. The present disclosure ameliorates the onset of idiopathic RBD and is expected to delay the risk of developing neurodegenerative diseases such as PD or dementia with Lewy body in iRBD patients.

### Efficacy Trial 3 of the Present Disclosure—Idiopathic RBD (iRBD) Efficacy Data

### I. Materials and Methods

a) Subjects

1. Inclusion criteria:

Inclusion criteria for patient with idiopathic RBD (iRBD)

1) Those who were aged 40 to 85 years old, male or female;

2) For Rapid eye movement sleep behavior disorder screening questionnaire (RBDSQ), RBDSQ >5, and for Rapid eye movement sleep behavior disorder questionnaire Hong Kong (RBD-HK for short), RBD-HK score ≥17; and

3) Diagnostic criteria for iRBD: Diagnosis was confirmed by a physician at Department of Neurology & Sleep Disorders Center based on polysomnography (PSG for short), where the patient had a history of dream enactment behavior, and video polysomnography showed increased electromyography activity or abnormal behavior during the rapid eye movement.

2. Exclusion criteria:

1) Those with a history of serious mental illness or drug abuse; a Hamilton Depression Scale score of ≥17, and/or an anxiety scale score of ≥14 or being treated with oral antidepressant or antipsychotics; being treated with oral clonazepam or other benzodiazepines and/or melatonin within 4 weeks; and

2) Those who had other central nervous system diseases, including cerebrovascular disease, brain tumors, inflammation, genetic metabolic diseases, traumatic brain injury, definite degenerative diseases of the central nervous system (e.g. Parkinson's disease, multiple system atrophy, dementia with Lewy body, etc.), and the like.

3. Randomization scheme:

This study was a prospective open cohort study. Patients were divided into test group or control group according to the order of their visits combined with their wishes.

4. Drop out, discontinuation and withdrawal scheme:

Patients who cannot continue to participate in this study, or patients who cannot be contacted during multiple follow-up are considered as drop out; those who developed any inflammation required to be treated with antibiotics during the observation period will have unaccepted scale scores during the use period and are considered as discontinuation; and those who developed acute cardiovascular and cerebrovascular accidents or other serious adverse events are considered as withdrawal.

### II. Test Method

a) Intervention samples:

Test group: the probiotic formulation was obtained in Example 4 of the present disclosure.

Control group: only pre-enrollment therapy thereof was maintained, and no probiotics or prebiotics were added.

b) Test method:

The test group was given the formulation of Example 4, 2 capsules each time, three times a day. The control group was not given any RBD-related treatment.

c) Evaluation: All patients were collected for electrocardiogram, blood routine, liver and kidney function, and electrolyte testing at the time of enrollment and week 12. Rapid eye movement sleep behavior disorder screening questionnaire (RBDSQ) was used for screening, and rapid eye movement sleep behavior disorder questionnaire Hong Kong (RBD-HK) was used to assess RBD and the severity thereof.

d) Data statistics: SPSS26.0 was used for statistical analysis of the data. Measurement data were described as X±SE, an independent sample t-test was used to compare the data between groups, a paired sample t-test was used to compare the data within the group, α was set as 0.05, and P<0.05 meant that the difference was statistically significant.

### III. Test Results and Analysis

### General Patient Information

A total of 15 iRBD patients who attended at Beijing Friendship Hospital, Capital Medical University from March 2020 to March 2022, met the inclusion criteria and did not met the exclusion criteria were collected, including 10 in test group and 5 in control group; and there were 8 males and 2 females in test group, with a mean age of 68.40±2.418 years, and 2 males and 3 females in control group, with a mean age of 66.20±0.800 years. The RBDSQ scores of all patients were >5.

The RBD-HK score at baseline was 51.60±3.673 in test group and 57.60±8.658 in control group, with no statistically significant difference between the two groups (P=0.462, P>0.05). The control group had an RBD-HK score of 52.20±6.422 at week 12, which was not statistically different from baseline (P=0.588, P>0.05). The test group had an RBD-HK score of 37.60±3.423 at week 12, which was statistically significantly different from baseline (P<0.001), and statistically significantly different from the control group in the RBD-HK score at week 12 (P=0.045, P<0.05).

TABLE 9
RBD-HK scores of iRBD patients in both groups
before and after intervention (score, X ± SE)

| Group | Week 0 | Week 12 |
|---|---|---|
| Control group | 57.60 ± 8.658 | 52.20 ± 6.422 |
| (n = 5) | | |
| Test group | 51.60 ± 3.673 | 37.60 ± 3.423***^{#} |
| (n = 10) | | |

*P < 0.05,
**P < 0.01, and
***P < 0.001, compared to before treatment; and
^{#}P < 0.05,
## P < 0.01, and
### P < 0.001, compared to the same period in control group.

FIG. 9 shows the comparison of RBD-HK scores before and after the iRBD intervention in both groups. *P<0.05, **P<0.01, and ***P<0.001, compared to before treatment; and # P<0.05, ## P<0.01, and ### P<0.001, compared to the same period in control group.

### IV. Description of the Test Results

Idiopathic rapid eye movement sleep behavior disorder (iRBD) is currently considered to be an important biomarker of the prodromal phase in Parkinson's disease. A prospective open cohort study of randomly enrolled patients with iRBD was conducted and it was found that the probiotic composition could significantly improve the RBD-HK score in iRBD patients. Combined with the results of studies in PD patients, the probiotic composition was shown to have a significant improvement in both RBD in patients with Parkinson's disease and idiopathic RBD. It is suggested that the probiotic composition can be applied in the prodromal phase of Parkinson's disease and is expected to delay the conversion of the prodromal phase to the clinical phase in Parkinson's disease.

What is claimed is:

1. A probiotic composition for treating rapid eye movement sleep behavior disorder, comprising Bacillus licheniformis, Bifidobacterium longum, Lactobacillus acidophilus, and Enterococcus faecalis.

2. The probiotic composition for treating rapid eye movement sleep behavior disorder according to claim 1, further comprising adjuvants.

3. The probiotic composition for treating rapid eye movement sleep behavior disorder according to claim 1, wherein
the viable count of Bacillus licheniformis in the probiotic composition is not less than 1×10⁷ CFU/g.

4. The probiotic composition for treating rapid eye movement sleep behavior disorder according to claim 1, wherein
the viable count of Bifidobacterium longum in the probiotic composition is not less than 1×10⁷ CFU/g.

5. The probiotic composition for treating rapid eye movement sleep behavior disorder according to claim 1, wherein
the viable count of Lactobacillus acidophilus in the probiotic composition is not less than 1×10⁷ CFU/g.

6. The probiotic composition for treating rapid eye movement sleep behavior disorder according to claim 1, wherein
the viable count of Enterococcus faecalis in the probiotic composition is not less than 1×10⁷ CFU/g.

7. The probiotic composition for treating rapid eye movement sleep behavior disorder according to claim 1, wherein the probiotic composition is obtained by the following steps:
preparing the probiotic composition according to claim 1 as a lyophilized powder and then mixing.

8. The probiotic composition for treating rapid eye movement sleep behavior disorder according to claim 1, wherein
the rapid eye movement sleep behavior disorder comprises idiopathic rapid eye movement sleep behavior disorder and/or rapid eye movement sleep behavior disorder in Parkinson's disease.

9. A method for treating rapid eye movement sleep behavior disorder by administration of a probiotic composition, wherein
the probiotic composition is prepared from Bacillus licheniformis, Bifidobacterium longum, Lactobacillus acidophilus, and Enterococcus faecalis as well as adjuvants.

10. The method according to claim 9, wherein
the viable count of Bacillus licheniformis in the probiotic composition is not less than 1×10⁷ CFU/g.

11. The method according to claim 9, wherein
the viable count of Bifidobacterium longum in the probiotic composition is not less than 1×10⁷ CFU/g.

12. The method according to claim 9, wherein
the viable count of Lactobacillus acidophilus in the probiotic composition is not less than 1×10⁷ CFU/g.

13. The method according to claim 9, wherein
the viable count of Enterococcus faecalis in the probiotic composition is not less than 1×10⁷ CFU/g.

14. The method according to claim 9, wherein
the probiotic composition can be prepared as probiotic formulations, drugs, nutraceuticals and/or food products.

15. A probiotic formulation for treating rapid eye movement sleep behavior disorder,
comprising Bacillus licheniformis, Bifidobacterium longum, Lactobacillus acidophilus, and Enterococcus faecalis as well as adjuvants used for preparing the probiotic composition as a probiotic dosage form.

16. The probiotic formulation according to claim 15, wherein
the viable count of Bacillus licheniformis in the probiotic composition is not less than 1×10⁷ CFU/g;
the viable count of Bifidobacterium longum in the probiotic composition is not less than 1×10⁷ CFU/g;
the viable count of Lactobacillus acidophilus in the probiotic composition is not less than 1×10⁷ CFU/g; and
the viable count of Enterococcus faecalis in the probiotic composition is not less than 1×10⁷ CFU/g.

17. The probiotic formulation according to claim 15, wherein the probiotic formulation is obtained by the following steps:
(1) preparing each bacterial powder: preparing dried bacterial powder of the Bacillus licheniformis, Bifidobacterium longum, Lactobacillus acidophilus, and Enterococcus faecalis, respectively; and
(2) mixing each dried bacterial powder obtained in step (1) with the adjuvants, forming and dispensing.

18. A method for treating rapid eye movement sleep behavior disorder by administration of the probiotic formulation according to claim 15.

19. A food composition or supplement comprising a probiotic composition, wherein the probiotic composition is prepared from Bacillus licheniformis, Bifidobacterium longum, Lactobacillus acidophilus, and Enterococcus faecalis, as well as adjuvants.

20. The food composition or supplement according to claim 19, wherein
the viable count of Bacillus licheniformis in the probiotic composition is not less than 1×10⁷ CFU/g;
the viable count of Bifidobacterium longum in the probiotic composition is not less than 1×10⁷ CFU/g;
the viable count of Lactobacillus acidophilus in the probiotic composition is not less than 1×10⁷ CFU/g; and
the viable count of Enterococcus faecalis in the probiotic composition is not less than 1×10⁷ CFU/g.
